(19) United States
(12) Patent Application Publication
(10) Pub. No.: US 2022/0392627 A1
(43) Pub. Date: Dec. 8, 2022

(54) ANOMALY DETECTION FOR SENSED ELECTROPHYSIOLOGICAL DATA

(71) Applicant: Boston Scientific Neuromodulation Corporation, Valencia, CA (US)

(72) Inventors: Andrew James Haddock, Los Angeles, CA (US); Adarsh Jayakumar, Valencia, CA (US)

(21) Appl. No.: 17/833,538

(22) Filed: Jun. 6, 2022

Related U.S. Application Data
(60) Provisional application No. 63/197,612, filed on Jun. 7, 2021.

(51) Int. Cl. G16H 40/63

(52) U.S. Cl. CPC G16H 40/63

(57) ABSTRACT

A system may include a stimulator, sensing circuitry and a controller. The stimulator may be configured to deliver an electrical therapy using at least one electrode by delivering an electrical waveform according to waveform parameters. The sensing circuitry may be configured to sense electrical potentials. A controller may be configured to detect at least one feature in the sensed electrical potentials, provide closed-loop control of the stimulator using a control algorithm and the detected at least one feature as an input into the control algorithm, determine whether the detected at least one feature is anomalous with respect to the feature data used to determine the one or more relationships, and perform remedial action when it is determined that the at least one feature is anomalous with respect to the feature data.

### CLAIM OF PRIORITY

This application claims the benefit of U.S. Provisional Application No. 63/197,612, filed on Jun. 7, 2021, which is hereby incorporated by reference in its entirety.

### TECHNICAL FIELD

This document relates generally to medical systems, and more particularly, but not by way of limitation, to systems, devices, and methods for detecting anomalies in sensed electrophysiological data.

### BACKGROUND

Various therapies may deliver electrical energy to a patient. Examples of such therapies include, but are not limited to, muscle stimulators, cardiac rhythm devices such as pacemakers and defibrillators, and neurostimulators. Physiological signal(s) may be sensed for various reasons related to the delivered therapy, such as to time the therapy delivery, to determine enabling or disabling conditions for delivering the therapy, to determine an efficacy of a therapy, or to provide feedback for closed-loop control of the therapy. For example, action potentials within a nerve may be sensed to provide closed-loop control of a neuromodulation therapy. Examples of neuromodulation include Spinal Cord Stimulation (SCS), Deep Brain Stimulation (DBS), Peripheral Nerve Stimulation (PNS), and Functional Electrical Stimulation (FES).

### SUMMARY

An example (e.g., “Example 1”) of a system may include a stimulator, sensing circuitry and a controller. The stimulator may be operably connected to at least one stimulation electrode, and configured to deliver an electrical therapy using the at least one electrode by delivering an electrical waveform according to waveform parameters. The sensing circuitry may be operably connected to at least one sensing electrode, and configured to sense electrical potentials. A controller may be operably connected to the stimulator and the sensing circuitry. The controller may be configured to detect at least one feature in the sensed electrical potentials, provide closed-loop control of the stimulator using a control algorithm and the detected at least one feature as an input into the control algorithm, determine whether the detected at least one feature is anomalous with respect to the feature data used to determine the one or more relationships, and perform remedial action when it is determined that the at least one feature is anomalous with respect to the feature data. The control algorithm may define one or more relationships between at least one feature and the one or more of the waveform parameters, the one or more relationships being determined using feature data.

In Example 2, the subject matter of Example 1 may optionally be configured such that the sensed electrical potentials may include local field potentials, evoked compound action potentials (ECAPs), or evoked resonant neural activity (ERNA).

In Example 3, the subject matter of Example 2 may optionally be configured such that the sensed electrical potentials may include neural activity or muscle activity.

In Example 4, the subject matter of any one or more of Examples 1-3 may optionally be configured such that the at least one feature may include: at least one peak, the at least one peak including a minimum peak, a maximum peak, a local minimum peak or a local maximum peak; an area under a curve; a curve length; an oscillation frequency; or a rate of decay for a peak amplitude.

In Example 5, the subject matter of any one or more of Examples 1-4 may optionally be configured such that the controller may be configured to provide closed-loop control of the stimulator based on a feature change for the detected at least one feature with respect to a baseline or a feature difference.

In Example 6, the subject matter of any one or more of Examples 1-5 may optionally be configured such that the system may be configured to implement unsupervised machine learning techniques to determine whether the detected at least one feature is anomalous with respect to the feature data. The unsupervised machine learning techniques may include a density-based supervised clustering of apps with noise (DBSCAN) or an isolation forest.

In Example 7, the subject matter of any one or more of Examples 1-6 may optionally be configured such that the controller may be configured to use statistical analysis to determine that the detected at least one feature is anomalous with respect to the feature data.

In Example 8, the subject matter of Example 7 may optionally be configured such that the detected at least one feature is quantified using digits, and the statistical analysis may include analyzing a most significant digit for the quantified value using Benford's law.

In Example 9, the subject matter of any one or more of Examples 1-8 may optionally be configured such that the statistical analysis may include a Z-score calculated as a difference between a data point for the at least one feature and a mean of the training data. The mean is divided by a standard deviation of the training data, and the at least one feature may be determined to be anomalous when the Z-score exceeds a threshold.

In Example 10, the subject matter of any one or more of Examples 1-9 may optionally be configured such that the statistical analysis may include a boxplot derived from training data. The at least one feature may be determined to be anomalous when a data point for the at least on feature is great than or less than a factor of an upper limit for an interquartile range or a factor of a lower limit for the interquartile range.

In Example 11, the subject matter of any one or more of Examples 1-10 may optionally be configured such that the controller may be configured to determine whether the detected at least one feature is anomalous before detecting a subsequent instance of the at least one feature in the sensed evoked signal.

In Example 12, the subject matter of any one or more of Examples 1-10 may optionally be configured such that the controller may be configured to determine that the detected at least one feature is not anomalous before adjusting at least one waveform parameter based on the detected at least one feature.

In Example 13, the subject matter of any one or more of Examples 1-10 may optionally be configured such that the controller is configured to store a plurality of instances of the detected at least one feature, and audit the plurality of instances to determine if any one or more of the instances correspond to anomalous detected at least one feature.

In Example 14, the subject matter of any one or more of Examples 1-13 may optionally be configured such that the remedial action may include: disabling or adjusting the closed-loop control; reconfiguring a sensing configuration; or reconfiguring the feature data used to determine the one or more relationships between the at least one feature and the one or more waveform parameters.

In Example 15, the subject matter of any one or more of Examples 1-14 may optionally be configured such that the remedial action may include communicating with the patient to troubleshoot or to send a report.

Example 16 includes subject matter (such as a method, means for performing acts, machine readable medium including instructions that when performed by a machine cause the machine to performs acts, or an apparatus to perform). The subject matter may include delivering an electrical therapy using a stimulator operably connected to at least one electrode by delivering an electrical waveform according to waveform parameters, sensing electrical potentials using sensing circuitry, and using a controller to automatically perform a process. The automatically performed process may include detecting at least one feature in the sensed electrical potentials, providing closed-loop control of the stimulator using a control algorithm and the detected at least one feature as an input into the control algorithm. The control algorithm may define one or more relationships between at least one feature and the one or more of the waveform parameters. The one or more relationships may be determined using feature data. The control algorithm may determine whether the detected at least one feature is anomalous with respect to the feature data used to determine the one or more relationships. The control algorithm may perform remedial action when it is determined that the at least one feature is anomalous with respect to the feature data.

In Example 17, the subject matter of Example 16 may optionally be configured such that the sensing electrical potentials may include sensing local field potentials, evoked compound action potentials (ECAPs), or evoked resonant neural activity (ERNA).

In Example 18, the subject matter of Example 16 may optionally be configured such that the sensing electrical potentials may include sensing neural activity or sensing muscle activity.

In Example 19, the subject matter of any one or more of Examples 16-18 may optionally be configured such that the detecting at least one feature may include: detecting at least one peak, the at least one peak including a minimum peak, a maximum peak, a local minimum peak or a local maximum peak; detecting an area under a curve; detecting a curve length; detecting an oscillation frequency; or detecting a rate of decay for a peak amplitude.

In Example 20, the subject matter of any one or more of Examples 16-19 may optionally be configured such that the providing closed-loop control may include providing closed-loop control based on a feature change for the detected at least one feature with respect to a baseline or a feature difference.

In Example 21, the subject matter of any one or more of Examples 16-20 may optionally be configured to further include implementing unsupervised machine learning techniques to determine whether the detected at least one feature is anomalous with respect to the feature data. The unsupervised machine learning techniques may include a density-based supervised clustering of apps with noise (DBSCAN) or an isolation forest.

In Example 22, the subject matter of any one or more of Examples 16-21 may optionally be configured such that the determining whether the detected at least one feature is anomalous may include performing statistical analysis to determine that the detected at least one feature is anomalous with respect to the feature data.

In Example 23, the subject matter of any one or more of Example 22 may optionally be configured such that the detected at least one feature is quantified using digits, and the statistical analysis includes analyzing a most significant digit for the quantified value using Benford's law.

In Example 24, the subject matter of any one or more of Examples 16-23 may optionally be configured such that the statistical analysis may include a Z-score calculated as a difference between a data point for the at least one feature and a mean of the training data. The mean may be divided by a standard deviation of the training data. The at least one feature may be determined to be anomalous when the Z-score exceeds a threshold.

In Example 25, the subject matter of any one or more of Examples 16-24 may optionally be configured such that the statistical analysis may include a boxplot derived from training data. The at least one feature may be determined to be anomalous when a data point for the at least on feature is great than or less than a factor of an upper limit for an interquartile range or a factor of a lower limit for the interquartile range.

In Example 26, the subject matter of any one or more of Examples 16-25 may optionally be configured such that the determining whether the detected at least one feature is anomalous may be performed before detecting a subsequence instance of the at least one feature in the sensed evoked signal.

In Example 27, the subject matter of any one or more of Examples 16-25 may optionally be configured such that the detected at least one feature may be determined to be not anomalous before adjusting at least one waveform parameter based on the detected at least one feature.

In Example 28, the subject matter of any one or more of Examples 16-25 may optionally be configured to further include storing a plurality of instances of the detected at least one feature, and auditing the plurality of instances to determine if any one or more of the instances correspond to anomalous detected at least one feature.

In Example 29, the subject matter of any one or more of Examples 16-28 may optionally be configured such that the performing remedial action may include automatic and/or manual processes for: disabling or adjusting the closed-loop control; reconfiguring a sensing configuration; or reconfiguring the feature data used to determine the one or more relationships between the at least one feature and the one or more waveform parameters.

In Example 30, the subject matter of any one or more of Examples 16-29 may optionally be configured such that the performing remedial action may include communicating with the patient to troubleshoot or to send an encrypted report.

Example 31 includes subject matter (such as a device, apparatus, or machine) that may include non-transitory machine-readable medium including instructions, which when executed by a machine, cause the machine to perform a method comprising delivering an electrical therapy by delivering an electrical waveform according to waveform parameters; sensing electrical potentials; and automatically perform a process. The automatically performed process may include: detecting at least one feature in the sensed electrical potentials; providing closed-loop control of the stimulator using a control algorithm and the detected at least one feature as an input into the control algorithm. The control algorithm may define one or more relationships between at least one feature and the one or more of the waveform parameters. The one or more relationships may be determined using feature data. The control algorithm may determine whether the detected at least one feature is anomalous with respect to the feature data used to determine the one or more relationships. The control algorithm may perform remedial action when it is determined that the at least one feature is anomalous with respect to the feature data.

In Example 32, the subject matter of Example 31 may optionally be configured such that the detected at least one feature is quantified using digits, and the statistical analysis includes analyzing a most significant digit for the quantified value using Benford's law.

In Example 33, the subject matter of any one or more of Examples 31-32 may optionally be configured such that the statistical analysis may include a Z-score calculated as a difference between a data point for the at least one feature and a mean of the training data. The mean may be divided by a standard deviation of the training data. The at least one feature may be determined to be anomalous when the Z-score exceeds a threshold.

In Example 34, the subject matter of any one or more of Examples 31-33 may optionally be configured such that the statistical analysis may include a boxplot derived from training data. The at least one feature may be determined to be anomalous when a data point for the at least on feature is great than or less than a factor of an upper limit for an interquartile range or a factor of a lower limit for the interquartile range.

In Example 35, the subject matter of any one or more of Examples 31-34 may optionally be configured such that the performing remedial action may include disabling the closed-loop control, or communicating with the patient to troubleshoot or to send a report.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates, by way of example, an embodiment of a neuromodulation system.

FIG. 2 illustrates, by way of example and not limitation, the neuromodulation system of FIG. 1 implemented in a spinal cord stimulation (SCS) system or a deep brain stimulation (DBS) system.

FIG. 3 illustrates, by way of example and not limitation, a method for providing closed-loop modulation, based on training data, using sensed electrical activity as feedback.

FIG. 4 illustrates, by way of example and not limitation, training and use of a machine-learning program, according to some example embodiments.

FIG. 5 illustrates, by way of example and not limitation, a process for implementing a closed-loop therapy using a trained algorithm to control waveform parameter(s) according to signal feature feedback, including detecting anomalous feature(s) with respect to feature data used to develop the trained algorithm.

FIG. 6 illustrates, by way of example and not limitation, anomaly detection timing such as may be performed by a neuromodulator in real-time or near-real-time, in which anomaly detection is performed for each instance (e.g., epoch) of detected feature(s).

FIG. 7 illustrates, by way of example and not limitation, anomaly detection timing such as may be performed by a neuromodulator in real-time or near-real-time, in which anomaly detection is performed before adjusting waveform parameter(s) .

FIG. 8 illustrates, by way of example and not limitation, anomaly detection timing such as may be performed by a neuromodulator system, in which data relied upon for controlling the closed-loop therapy is evaluated for anomalies.

FIG. 9 illustrates, by way of example and not limitation, a control loop for detecting anomalies such as may occur in real-time or near-real time.

FIG. 10 illustrates, by way of example and not limitation, an audit process for detecting anomalies in sensed data.

FIG. 11 illustrates, by way of example and not limitation, a process for performing remedial action(s) in response to detecting an anomaly or multiple anomalies.

FIG. 12 illustrates, by way of example and not limitation, an embodiment of a modulation device, such as may be implemented in the neuromodulation system of FIG. 1, that includes anomaly detection of sensed data.

FIG. 13 is a diagram illustrating a relationship between a stimulation electrode and a sensing electrode.

FIG. 14 illustrates a statistical approach for detecting anomalies that uses a boxplot derived from training data.

FIG. 15 illustrates an unsupervised learning approach for performing a data audit on stored feature data and/or signal samples using a density-based supervised clustering of apps with noise (DBSCAN).

FIG. 16 illustrates an unsupervised learning approach for performing a data audit on stored feature data and/or signal samples using an isolation forest technique.

FIG. 17 illustrates a technique based on an empirical law, such as Benford's law, for performing a data audit on stored feature data and/or signal samples.

FIGS. 18-22 illustrate, by way of example and not limitation, clinical data following Benford's law, where FIG. 18 illustrates a local field potential (LFP) from a monkey's motor cortex, FIG. 19 illustrates the Benford's curve as well as the most significant bit of the LFP data provided in FIG. 18, FIG. 20 illustrates evoked compound action potential (ECAP) data, FIG. 21 illustrates the Benford's curve as well as the most significant bit for different channels of the ECAP data provided in FIG. 20, and FIG. 22 illustrates the most significant bit for different channels of the LFP data.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

A system may be configured to sense electrophysiological signals for reasons such as to time the therapy delivery, to determine enabling or disabling conditions for delivering the therapy, to determine an efficacy of a therapy, or to provide feedback for closed-loop control of the therapy. By determining when the sensed data is abnormal or anomalous, the present subject matter may validate the accuracy of the sensed data, and thus ensure the value of the sensed data to the system.

For example, a system configured to deliver a therapy, such as an electrical therapy, may use sensed electrophysiological signals as feedback for use to enable closed-loop control of the therapy. An example of an electrical therapy is neuromodulation therapy. Neuromodulation therapies may include, by way of example and not limitation, SCS, DBS, PNS, or FES therapies. The system may be configured to implement closed-loop algorithms that use the sensed electrophysiological signals or data determined from the signals to regulate or optimize the therapy. For example, the relationship(s) between stimulation parameter(s) (e.g., neuromodulation parameter(s) such as SCS, DBS, PNS or FES parameter(s) and feature(s) extracted from the sensed electrophysiological data may be determined and encoded using training data. These relationship(s) may be used to determine the closed-loop algorithms that are used to control the therapy.

However, these relationship(s) between the stimulation parameter(s) and extracted feature(s) may be altered, which may render the closed-loop algorithm(s) ineffective or incapable of producing desired effects. For example, environmental effects, such as lead migration, impedance changes, scar tissue formation, disease progression, electromagnetic interference or other disturbances on both short and long-time scales, can alter the relationship between the neuromodulation parameter(s) and extracted feature(s). The training data used to determine the closed-loop algorithm may become outdated when these relationships change.

In order to minimize the occurrence and duration of ineffective or undesired neuromodulation therapy during closed-loop therapies, the present subject matter may detect anomalies in the sensed signal. Anomalies deviate from what is standard, normal or expected. Statistical techniques may be used to correctly classify data as anomalous or within the expected range. For example, the detection of anomalies may include real-time (the time during which the process takes place) or near-real-time anomaly detection and/or data auditing. Real-time indicates almost immediate detection as the process is taking place, and near-real-time indicates that there is a delay that may be associated with data processing and communication, but the delays are not significant delays for process timing. The present subject matter may perform a remedial action when it has been determined that there have been one or more anomalies. For example, the present subject matter may take actions to interrupt an ongoing closed-loop therapy, prompt manual or automatic collection of new training data, and/or otherwise relay error messages to patients, clinicians, field representatives, or other users of the system. For example, stimulation parameters in a model-based design may be updated when it has been determined that sensed electrophysiological data is anomalous and may cause unstable system behavior. The system may be designed to monitor and regulate closed-loop algorithm behavior and ensure that any deficiencies are promptly addressed and solved, such as, by way of example and not limitation, by reverting to a fallback mode and prompt the user for engagement to reset the closed-loop system. The user may be presented with troubleshooting steps when an anomaly is sensed.

An embodiment implements an anomaly detector in firmware of a neuromodulator, where the anomaly detector performs a relatively simple detection of anomalies on feature data performed in real time or near real time as the feature data is determined. For example, the anomaly detector may look for anomalies every epoch. An epoch is a relatively short period of time, such that the anomaly detection is in real-time or near-real-time. For example, anomaly detection may be performed each time or after more than one time that the system senses or detect features, each stimulation epoch or after more than one stimulation epochs, or each control epoch or more than one control epoch. A stimulation epoch is a period of time between stimulation pulses, and a control epoch is a period of time when the programmed control algorithm executes and updates stimulation. The control epoch and the stimulation epoch may be the same, or the control epoch could span two or more stimulation epochs (e.g., a control epoch corresponds to five stimulation pulses). The real-time or near-real-time anomaly detector may be computationally efficient to detect anomalies with single epoch. By way of example, simple statistical approaches like Z-scoring may be used to detect anomalies.

An embodiment may audit stored feature data (or stored signals) every time period (e.g., on the order of hours, days, or weeks). The audit may be performed in a fallback mode. The audit may send data through a high-performance anomaly detector, capable of performing complex analysis, using large dataset storing a plurality of instances of detected feature(s) and/or signal samples. The system may use unsupervised learning methods and/or empirical laws (e.g., Benford's law) to audit the data. The system (e.g., firmware in the neuromodulator), may generate and store an anomaly report. The report may be sent to an external device, such as a connected app on a phone or other device via low energy Bluetooth (BLE).

According to various embodiments, the frequency of how often these algorithms (timing/count of instances or epochs) can be changed for an application without a firmware upgrade. The change may be automatic based on the application, or may be selected or changed by the patient or another user.

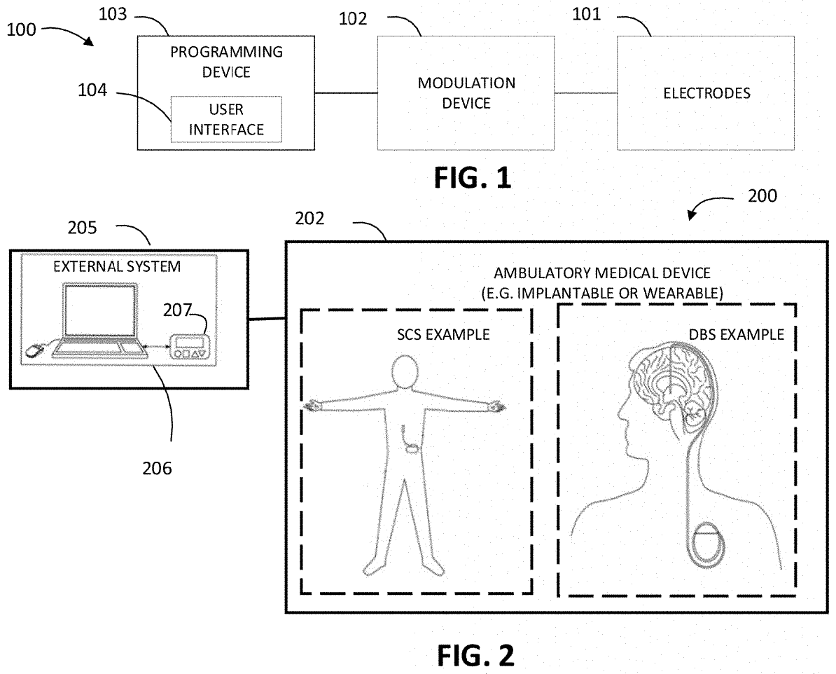

FIG. 1 illustrates, by way of example, an embodiment of a neuromodulation system. The illustrated neuromodulation system 100 includes electrodes 101, a modulation device 102, and a programming system such as a programming device 103. The programming system may include multiple devices. The electrodes 101 are configured to be placed on or near one or more neural targets in a patient. The modulation device 102 is configured to be electrically connected to electrodes 101 and deliver neuromodulation energy, such as in the form of electrical pulses, to the one or more neural targets though electrodes 101. The system may also include sensing circuitry to sense a physiological signal, which may but does not necessarily form a part of modulation device 102. The delivery of the neuromodulation is controlled using a plurality of modulation parameters that may specify the electrical waveform (e.g., pulses or pulse patterns or other waveform shapes) and a selection of electrodes through which the electrical waveform is delivered. In various embodiments, at least some parameters of the plurality of modulation parameters are programmable by a user, such as a physician or other caregiver. The programming device 103 provides the user with accessibility to the user-programmable parameters. The programming device 103 may also provide the use with data indicative of the sensed physiological signal or feature(s) of the sensed physiological signal. In various embodiments, the programming device 103 is configured to be communicatively coupled to modulation device via a wired or wireless link. In various embodiments, the programming device 103 includes a user interface 104 such as a graphical user interface (GUI) that allows the user to set and/or adjust values of the user-programmable modulation parameters. The user interface 104 may also allow the user to view the data indicative of the sensed physiological signal or feature(s) of the sensed physiological signal and may allow the user to interact with that data.

FIG. 2 illustrates, by way of example and not limitation, the neuromodulation system of FIG. 1 implemented in a spinal cord stimulation (SCS) system or a deep brain stimulation (DBS) system. The illustrated neuromodulation system 200 includes an external system 205 that may include at least one programming device. The illustrated external system 205 may include a clinician programmer 206 configured for use by a clinician to communicate with and program the neuromodulator, and a remote control 207 configured for use by the patient to communicate with and program the neuromodulator. For example, the remote control device may allow the patient to turn a therapy on and off and/or may allow the patient to adjust patient-programmable parameter(s) of the plurality of modulation parameters. FIG. 2 illustrates a modulation device as an ambulatory medical device 202. Examples of ambulatory devices include wearable or implantable neuromodulators. The external system 205 may include a network of computers, including computer(s) remotely located from the ambulatory medical device 202 that are capable of communicating via one or more communication networks with the programmer 206 and/or the remote control 207. The remotely located computer(s) and the ambulatory medical device 202 may be configured to communicate with each other via another external device such as the programmer 206 or the remote control 207.

The present subject matter may detect anomalies in the sensed signal, or in the data determined from the sensed signal. The data determined from the sensed signal may also be referred to as “extracted feature(s)” of the sensed signal. The anomaly detector may be implemented within the modulation device 102. For example, firmware with the modulation device 102 may detect the anomalies in the extracted feature(s).

The activities for detecting anomalies in the sensed electrical signal, or data determined from the sensed electrical signal, may be implemented in a single device or may be implemented using more than one device. The detection of anomalies may include real-time or near-real-time anomaly detection and/or data auditing. Anomaly detection may be implemented in software, firmware, or a combination thereof of at least one implantable device and/or at least one external device.

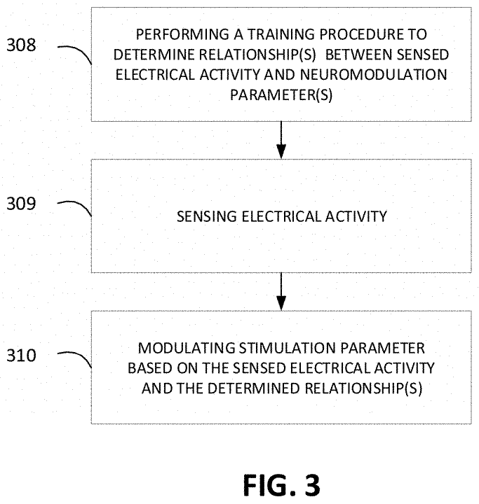

FIG. 3 illustrates, by way of example and not limitation, a method for providing closed-loop modulation, based on training data, using sensed electrical activity as feedback. The training data may be used by a machine learning algorithm to determine relationship(s) between the sensed electrical activity (e.g., extracted feature(s) of an electrical signal) and the parameter(s) of the neuromodulation. The method may include, at 308, performing a training procedure to determine a relationship between sensed electrical activity and neuromodulation parameters. Examples of sensed electrical activity includes neural activity or muscle activity. Examples include local field potentials, evoked compound action potentials (ECAPs), or evoked resonant neural activity (ERNA). For example, a training procedure may be performed by delivering the neurostimulation energy at one or more neurostimulation intensity levels to a neural target of the patient for evaluation. Feature(s) may be extracted from the sensed signal, and a relationship may be determined between the extracted feature(s) and the neuromodulation parameter(s) using mathematical or statistical modeling of the extracted feature(s). At 309, a physiological signal, such as electrical activity, is sensed, and stimulation parameters may be modulated according to the sensed electrical activity and the determined relationship, as illustrated at 310. Various stimulation parameters can be modulated, including but not limited to: current amplitude, frequency, pulse width, duty cycle, stimulation fractionalization, waveform shapes, waveform patterns (e.g. regular and/or irregular patterns of pulses or trains of pulses), stimulation on/off times, and combinations thereof.

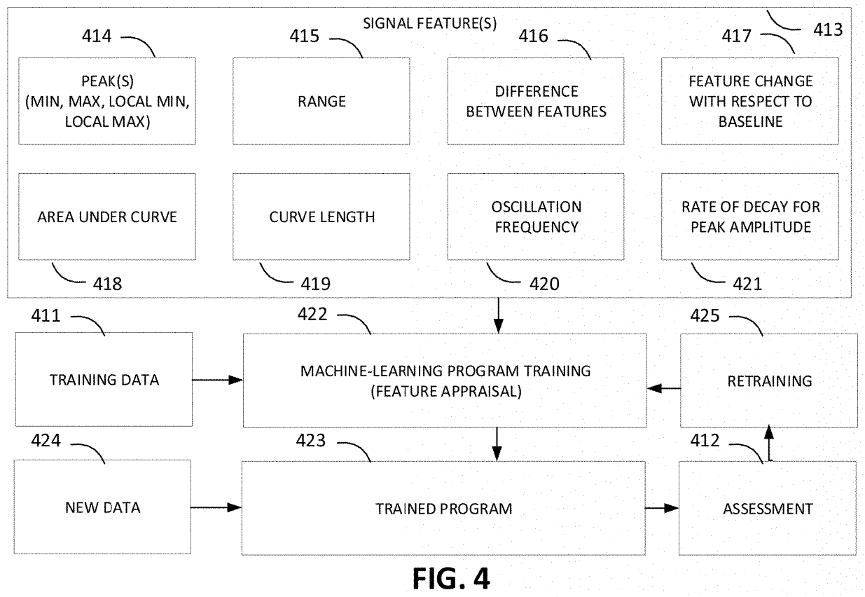

FIG. 4 illustrates, by way of example and not limitation, training and use of a machine-learning program, according to some example embodiments. In some example embodiments, machine-learning programs (MLPs), also referred to as machine-learning algorithms or tools, are utilized to perform operations associated with machine learning tasks, such as identifying relationship(s) between detected feature(s) in a sensed physiological signal and waveform parameter(s) used to control the neuromodulation.

Machine learning is a field of study that gives computers the ability to learn without being explicitly programmed. Machine learning explores the study and construction of algorithms that may learn from existing data (e.g., “training data”) and make predictions about new data. Such machine-learning tools may build a model from example training data 411 in order to make data-driven predictions or decisions expressed as outputs or assessments 412. The machine-learning algorithms use the training data 411 to find correlations among identified features 413 that affect the outcome.

The machine-learning algorithms use features 413 for analyzing the data to generate assessments 412. A feature is an individual measurable property of the observed phenomenon. In the context of a physiological signal, examples of features may include, but are not limited to, peak(s) 414 such as a minimum peak, a maximum peak as well as local minimum and maximum peaks, a range between peaks 415, a difference in values for features 416, a feature change with respect to a baseline 417, an area under a curve 418, a curve length 419, an oscillation frequency 420, and a rate of decay for peak amplitude 421. Inflection points in the signal may also be an observable feature of the signal, as an inflection point is a point where the signal changes concavity (e.g., from concave up to concave down, or vice versa), and may be identified by determining where the second derivative of the signal is zero. Detected feature(s) may be partially defined by time (e.g., length of curve over a time duration, area under a curve over a time duration, maximum or minimum peak within a time duration, etc.).

The machine-learning algorithms use the training data 411 to find correlations among the identified features 413 that affect the outcome or assessment 412. With the training data 411 and the identified features 413, the machine-learning tool is trained at operation 422. The machine-learning tool appraises the value of the features 413 as they correlate to the training data 411. The result of the training is the trained machine-learning program 423. Various machine learning techniques may be used to train models to make predictions based on data fed into the models. During a learning phase, the models are developed against a training dataset of inputs to optimize the models to correctly predict the output for a given input. A training data set may be defined for desired functionality of the closed-loop algorithm” and closed loop parameters may be defined for desired functionality of the closed-loop algorithm. Generally, the learning phase may be supervised, semi-supervised, or unsupervised; indicating a decreasing level to which the “correct” outputs are provided in correspondence to the training inputs. In a supervised learning phase, all of the outputs are provided to the model and the model is directed to develop a general rule or algorithm that maps the input to the output. In contrast, in an unsupervised learning phase, the desired output is not provided for the inputs so that the model may develop its own rules to discover relationships within the training dataset. In a semi-supervised learning phase, an incompletely labeled training set is provided, with some of the outputs known and some unknown for the training dataset.

Models may be run against a training dataset for several epochs (e.g., iterations), in which the training dataset is repeatedly fed into the model to refine its results. For example, in a supervised learning phase, a model is developed to predict the output for a given set of inputs, and is evaluated over several epochs to more reliably provide the output that is specified as corresponding to the given input for the greatest number of inputs for the training dataset. In another example, for an unsupervised learning phase, a model is developed to cluster the dataset into n groups, and is evaluated over several epochs as to how consistently it places a given input into a given group and how reliably it produces the n desired clusters across each epoch.

Once an epoch is run, the models are evaluated and the values of their variables are adjusted to attempt to better refine the model in an iterative fashion. In various aspects, the evaluations are biased against false negatives, biased against false positives, or evenly biased with respect to the overall accuracy of the model. The values may be adjusted in several ways depending on the machine learning technique used. For example, in a genetic or evolutionary algorithm, the values for the models that are most successful in predicting the desired outputs are used to develop values for models to use during the subsequent epoch, which may include random variation/mutation to provide additional data points. One of ordinary skill in the art will be familiar with several other machine learning algorithms that may be applied with the present disclosure, including linear regression, random forests, decision tree learning, neural networks, deep neural networks, etc.

New data 424 is provided as an input to the trained machine-learning program 422, and the trained machine-learning program 422 generates the assessment 412 as output. The outputted assessment 412 may be out of an expected range (e.g., anomalous), indicating that remedial action such as retraining 425 of the machine learning algorithm(s) is warranted. The system also may be configured to determine that the new data 424 includes anomalous data with respect to the training data 411 that was used to train the machine-learning program. The detection of new data that is anomalous may trigger remedial action(s) such as, if it is determined that the previously used training data is outdated, retraining 425 the machine learning program using updated training data.

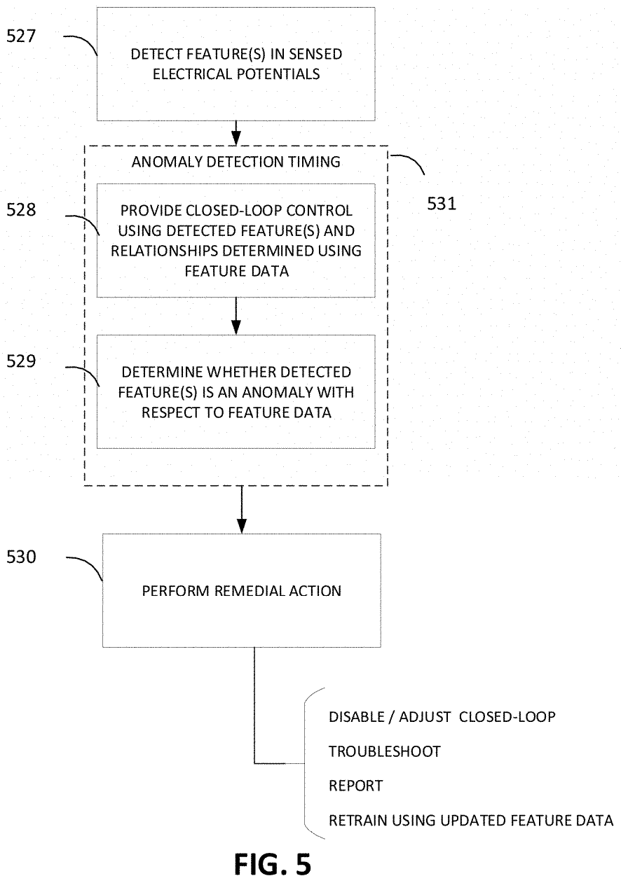

FIG. 5 illustrates, by way of example and not limitation, a process for implementing a closed-loop therapy using a trained algorithm to control waveform parameter(s) according to signal feature feedback, including detecting anomalous feature(s) with respect to feature data used to develop the trained algorithm. The illustrated process for implementing the closed-loop therapy includes detecting feature(s) within sensed electrical potentials, at 527, and providing closed-loop control using the detected feature(s) and relationship(s) determined using feature data (e.g., trained algorithm developed or trained based on the feature data) 528. The illustrated process may further include determining whether detected feature(s) is (are) an anomaly with respect to the feature data used to determine the relationship(s) 529. As illustrated at 530, remedial action may be performed upon determining that detected feature(s) is (are) anomalous with respect to the feature data used to develop the trained algorithm). By way of example and not limitation, the remedial action may include disabling the closed-loop control of the therapy. The therapy may be stopped completely, or may implement an open-loop therapy based on previously-determined waveform parameters. The remedial action may include adjusting the closed-loop therapy. The closed-loop therapy may be adjusted by adjusting the parameters and/or adjusting or replacing the algorithm itself. For example, the closed-loop algorithm may be adjusted by adjusting parameter value thresholds or functions (e.g., transfer function) implemented by the closed-loop algorithm to provide closed-loop control of the therapy. The remedial action may include presenting a troubleshooting question(s) and/or presenting reports to a patient or other user (e.g., via a remote control or other patient device (e.g., smartphone, a programmer, or a local or remote computer). The remedial action may include initiating a retraining of the trained algorithm using updated feature data to update the relationship(s) between detected feature(s) and waveform parameter(s). The remedial action may include manual and/or automatic activities. In some embodiments, the remedial action includes reconfiguring a sensing configuration. For example, signal processing such filtering, averaging, and/or sensing electrode selection may be changed as part of the remedial action.

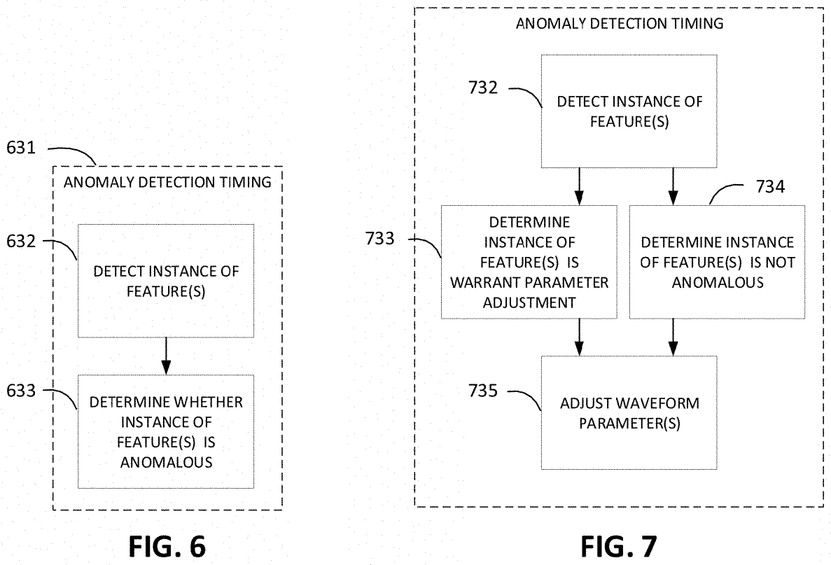

Various timing 531 for the anomaly detection may be implemented, some examples of which as generally illustrated in FIGS. 6-8. For example, the anomaly detection may be performed in real-time or in near real-time, or may be intermittently performed as an audit of stored feature data. For example, the anomaly detector may look for anomalies every epoch, such as a stimulation epoch between stimulation pulses or a control epoch between times when the programmed control algorithm executes and updates stimulation. The anomaly detector timing may use “instances” of detected features, which indicates times when a set of one or more feature(s) are extracted from a sensed signal. The instances of detected features may correspond to a control epoch, reflecting a period of time when the programmed control algorithm executes, analyzes sensor activity, and updates stimulation. However, a single control epoch may include one or more instances of detecting/extracting a set of one or more feature(s) in the sensed signal(s). The programmed control algorithm may be configured to use more than one feature detection instance to control the therapy. The anomaly detection may be configured to audit stored feature data (or stored signals) every time period (e.g., one the order of hours, days, or weeks). The time period may space across a plurality of instances of detected features or control epochs. The time period may be adjusted by the system or a user of the system.

The present subject matter may be implemented in a closed-loop spinal cord stimulation (SCS) application, by way of example and not limitation. The system may detect features that are greater than expected. This may be caused by lead movement (e.g., lead moving closer to a spinal cord) or may by increasing impedance of sensing contacts. The control algorithm may be adjusted to reduce the stimulation amplitude. If the extracted feature is more than the expected feature, then it may be determined that there has been an anomaly. The system may detect features that are less than expected. This may be caused by the lead moving further from the spinal cord, or from the impedance of the sensing contacts. The control algorithm may increase the stimulation amplitude. if the extracted feature is less than the expected feature, then it may be determined that there has been an anomaly. The control algorithm may be re-parameterized to account for shift in setpoints (feature vs. stimulation relationship). Automated detection and message reporting may be provided through firmware. A direct report may be provided to field representatives who then can manually re-program the neuromodulation device. The system may be configured to allow step the patient (or other user) through a troubleshooting routine with scripted action/questions.

FIG. 6 illustrates, by way of example and not limitation, anomaly detection timing 631 such as may be performed by a neuromodulator in real-time or near-real-time, in which anomaly detection is performed for each instance (e.g., epoch) of detected feature(s). For example, firmware within the neuromodulator may detect an instance of one or more features within the sensed electrical signal 632, and then at 633 determine whether the instance of feature(s) is anomalous with respect to the feature data used to determine the relationship(s) used to provide the closed-loop control. The firmware may check whether the instance is anomalous before proceeding to detect the next instance of feature(s).

By way of example and not limitation, the anomaly detector may use statistical testing, such as minimum values, maximum values and variance, on an extracted feature(s) to detect anomalies. The detected values may be compared with stimulation amplitude values for lower and upper bound checking. The data may be stored over a plurality of occurrences of instances for use in detecting trends. A failure or fallback mode maybe activated if a significant anomaly and/or performance degradation was detected. The control algorithm may have access to allowable stimulation parameters, the minimum and maximum amplitude, active stimulation parameters, an expected minimum and maximum, the variance and the value for each the feature(s) that is (are) being extracted,

FIG. 7 illustrates, by way of example and not limitation, anomaly detection timing 731 such as may be performed by a neuromodulator, in which anomaly detection is performed before adjusting waveform parameter(s). For example, firmware within the neuromodulator may detect, at 732, an instance of one or more features within the sensed electrical signal. After detecting an instance of feature(s), the firmware may determine, at 733, whether the instance of feature(s) warrant a waveform parameter adjustment as part of the closed-loop control of the therapy, and may determine that the instance of feature(s) is not anomalous at 734 before adjusting the waveform parameter(s) at 735.

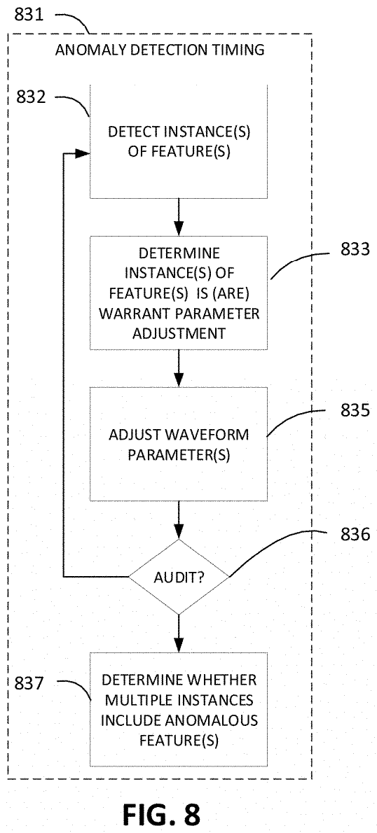

FIG. 8 illustrates, by way of example and not limitation, anomaly detection timing 831 such as may be performed by a neuromodulator system in which data relied upon for controlling the closed-loop therapy is evaluated for anomalies. For example, firmware within the neuromodulator may detect, at 832, at least one instance of one or more features within the sensed electrical signal. After detecting an instance of feature(s), the firmware may determine, at 833, whether the one or more instances of feature(s) warrant a waveform parameter adjustment as part of the closed-loop control of the therapy. The waveform parameters may be adjusted at 835. It may be determined at 836 whether to conduct an audit. If no audit is to be conducted, then the process may return to 832 to detect addition additional instance(s) of feature(s). If, at 836, it is determined that an audit is to be conducted, the process may evaluate multiple instance(s) of feature(s) to determine whether they include anomalous feature(s), as illustrated at 837.

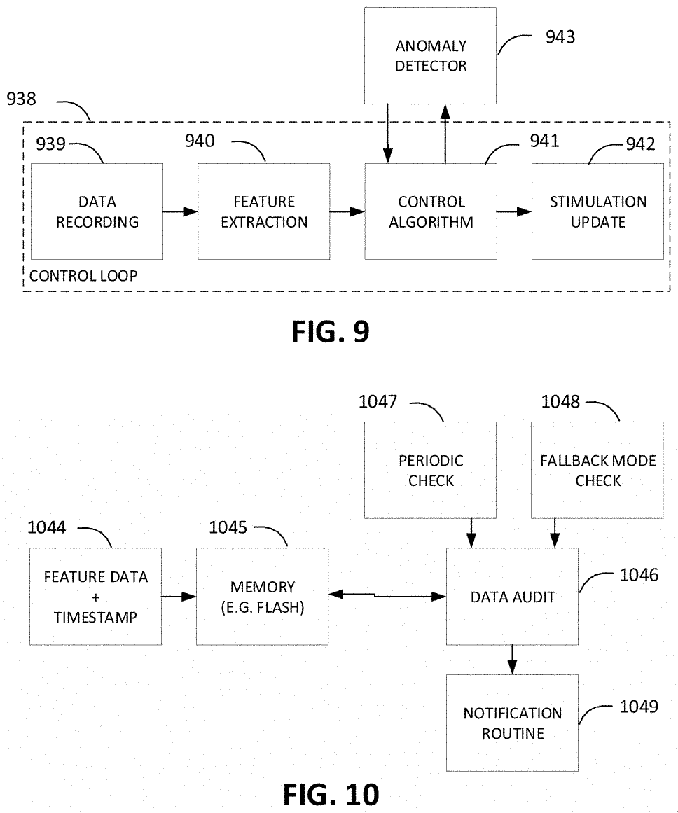

FIG. 9 illustrates, by way of example and not limitation, a control loop 938 for detecting anomalies such as may occur in real-time or near-real time. The control loop 938 generally includes data recording 939 (e.g., recorded signal data extracted from the sensed signal), and extracting feature(s) from the data recording 940. A control algorithm 941 updates the stimulation 942 based on the extracted feature(s). The anomaly detector 943 may be implemented with the control algorithm after control epochs.

FIG. 10 illustrates, by way of example and not limitation, an audit process for detecting anomalies in sensed data. Feature data and timestamps corresponding to the feature data 1044 may be stored in a memory 1045, such as a flash memory. The data audit 1046 may be performed on feature data stored in the memory. The data audit 1046 may be performed or as a periodic check. The period for the periodic check may be constant, may be variable, may be pre-programmed, or may be triggered by a user or sensed event. The data audit 1046 may be performed during a fallback mode 1048. For example, the system may enter a fallback mode when the anomalous data has been detected or anomalous outputs have been determined. At least some results of the performed data audit may be sent to user(s) via a notification routine 1049.

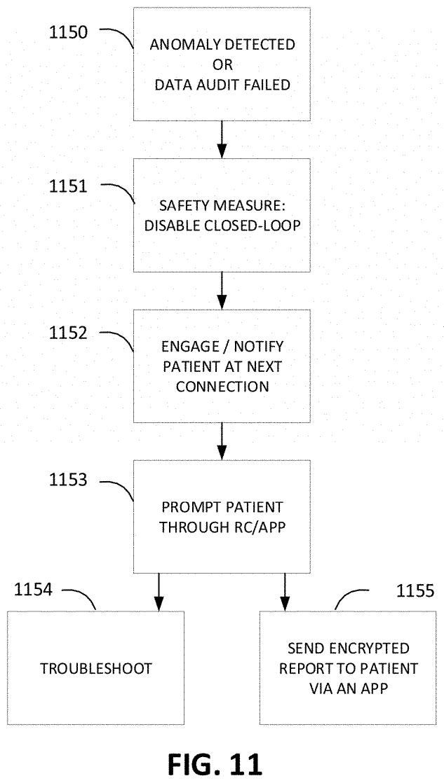

FIG. 11 illustrates, by way of example and not limitation, a process for performing remedial action(s) in response to detecting an anomaly or multiple anomalies. If an anomaly or anomalies have been detected or if a data audit failed as illustrated at 1150, the process may perform a safety measure such as disabling the closed-loop therapy or therapies 1151. Alternatively, the closed-loop algorithm may be adjusted or a different algorithm may be used that is not as susceptible to anomalous data. At 1152, the patient may be notified at the next connection with the implantable device. For example, the patient may be notified that the stimulation has been changed to open loop or to another fallback position. The patient may be prompted at 1153 for engagement (e.g., to troubleshoot the feature detection at 1154 or to send an encrypted report to the patient 1155). The patient may use a remote control (RC) and/or an app on a phone or tablet to receive the report. For example, firmware within the neuromodulator may generate and upload an encrypted anomaly report via Bluetooth Low Energy (BLE) through a connected app, and the app server may receive the anomaly report and send to field representatives. The anomaly report may include information regarding action(s) taken by the patient, such as, by way of examples and not limitation, who was notified of the anomaly, whether troubleshooting was attempted, troubleshooting activities, and whether a troubleshooting action was successful. An example of a troubleshooting action may be to instruct the patient to get into a position that known to the algorithm, and to use the RC/Aapp to find a comfortable amplitude for the stimulation at that position. When the comfortable amplitude is found, the user may press a start button to initiate a data collection procedure to retrain the closed-loop algorithm. If a comfortable amplitude is not able to be found, then it may be determined the problem may require other solutions such as solutions that may require a field representative such as reprogramming a fractionalization of energy contributions provided individual ones of the active electrodes.

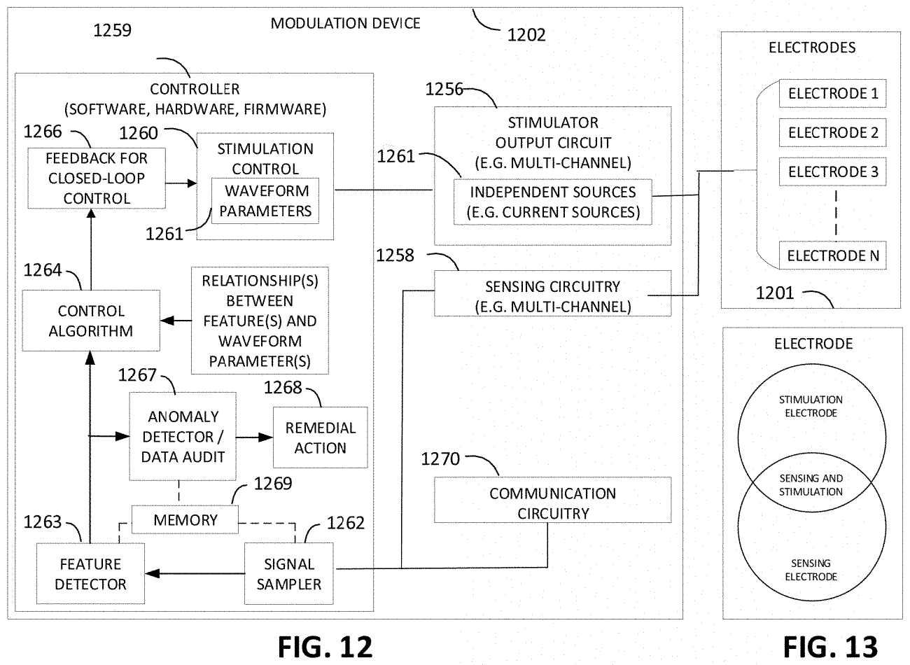

FIG. 12 illustrates, by way of example and not limitation, an embodiment of a modulation device, such as may be implemented in the neuromodulation system of FIG. 1, that includes anomaly detection of sensed data. The modulation device 1202 may be configured to be connected to electrode(s) 1201, illustrated as N electrodes. Any one or more of the electrodes 1201 may be configured for use to deliver modulation energy, sense electrical activity, or both deliver modulation energy and sense electrical activity. The modulation device 1202 may include a stimulator output circuit 1256 configured to deliver modulation energy to electrode(s). The stimulator output circuit 1256 may be configured with multiple (e.g., two or more) channels for delivering modulation energy, where each channel may be independently controlled with respect to other channel(s). For example, the stimulator output circuit 1256 may have independent sources 1257 such as independent current sources or independent voltage sources. The modulation device 1202 may include sensing circuitry 1258 configured to receive sensed electrical energy from the electrode(s), such as may be used to sense electrical activity in neural tissue or muscle tissue. The sensing circuitry may be configured to process signals in multiple (e.g., two or more) channels. By way of example and not limitation, the sensing circuitry 1258 may be configured to amplify and filter the signal(s) in the channel(s). Additionally or alternatively, the sensing circuitry may be configured for use with other types physiological sensors.

The modulation device 1202 may include a controller 1259 operably connected to the stimulation output circuit 1256 and the sensing circuitry 1258. The controller 1259 may include a stimulation control 1260 configured for controlling the stimulator output circuit 1256. For example, the stimulation control 1260 may include start/stop information for the stimulation and/or may include relative timing information between stimulation channels. The stimulation control 1260 may include waveform parameters 1261 that control the waveform characteristics of the waveform produced by the stimulation output circuit 1256. The waveform parameters 1261 may include, by way of example and not limitation, amplitude, frequency, and pulse width parameters. The waveform parameters 1261 may include, by way of example and not limitation, regular patterns such as patterns regularly repeat with same pulse-to-pulse interval and./or irregular patterns of pulses such as patterns with variable pulse-to-pulse intervals. The waveform parameters may, but do not necessary, define more than one waveform shape (e.g., including a shape other than square pulses with different widths or amplitudes). The stimulation control 1260 may be configured to change waveform parameter(s) (e.g., one or more waveform parameters) in response to user input and/or automatically in response to feedback.

The controller 1259 may include a signal sampler 1262 configured for use to sample a signal produced by the sensing circuitry 1258. The controller 1259 may further include a feature detector 1263 configured to detect one or more features in the sampled signal. Examples of features that may be detected include peaks (e.g., minimum and/or maximum peaks including local peaks/inflections), range between minimum/maximum peaks, local minima and/or local maxima, area under the curve (AUC), curve length between points in the curve, oscillation frequency, rate of decay after a peak, a difference between features, and a feature change with respect to a baseline. Detected feature(s) from the feature detector 1263 may be fed into a control algorithm 1264, which may use relationship(s) 1265 between the feature(s) and waveform parameter(s) to determine feedback for closed-loop control 1266 of the therapy. More than one algorithm may be used to provide the closed-loop control. The algorithm(s) may be selected from a plurality of algorithms that are available to be used to implement the closed-loop control. The different algorithms may use different feature(s) and/or control different waveform parameter(s), and/or have different transfer functions or sensitivity for adjusting the parameter(s) in response to changes in the feature(s). The closed-loop control 1266 may be used by the stimulation control 1260 to adjust the stimulation (e.g., parameter(s)). The controller 1259 of the modulation device 1202 may further include an anomaly detector 1267 configured to detect anomalies in the feature(s) detected by the feature detector 1263. These anomalies may be detected based on the feature data (e.g., training data) used to determine the relationship(s) between the feature(s) and the waveform parameter(s). The controller 1259 of the modulation device 1202 may further be configured to perform at least some activities for providing remedial action 1268 in response to a detected anomaly or detected anomalies. The controller 1259 may include a memory 1269 for storing the detected feature(s) and/or storing the sampled signals, for analysis in a data audit 1267 for detecting anomalies. Thus, the modulation device may be configured to detect anomalies in real-time or near-real-time using relatively simple and fast computation techniques, and/or may be configured to detect anomalies in stored data using more computationally-intensive anomaly detection techniques. The illustrated modulation device 1202 also include communication circuitry 1270 configured for use by the controller 1259 to communicate with other devices or systems (e.g., programmer or remote control) using one or more communication networks.

FIG. 13 is a diagram illustrating a relationship between a stimulation electrode and a sensing electrode. The stimulation electrode is configured for use in delivering modulation energy, and the sensing electrode is configured for use in sensing electrical activity. As illustrated by FIG. 12, the stimulation electrode may also be used in sensing electrical activity, and the sensing electrode may also be used in delivering modulation energy. Thus, the term “stimulation electrode” does not necessary exclude the electrode from also being used to sense electrical activity; and the term “sensing electrode” does not necessarily exclude the electrode from also being used to deliver modulation energy.

As provided above, anomalies may be detected in real-time or near-real-time using relatively simple and fast statistical techniques. One such statistical technique is the Z score. For example, both a standard deviation and a mean for values of a detected feature may be determined using training data. The z-score may be calculated as:

Z-score=(data point−mean)/standard deviation.

It may be determined that an anomaly has occurred if the absolute value of the z-score is greater than a threshold. This threshold may also be established during training data collection. The z-scoring (or other statistical technique) may be performed by firmware within the neuromodulation device.

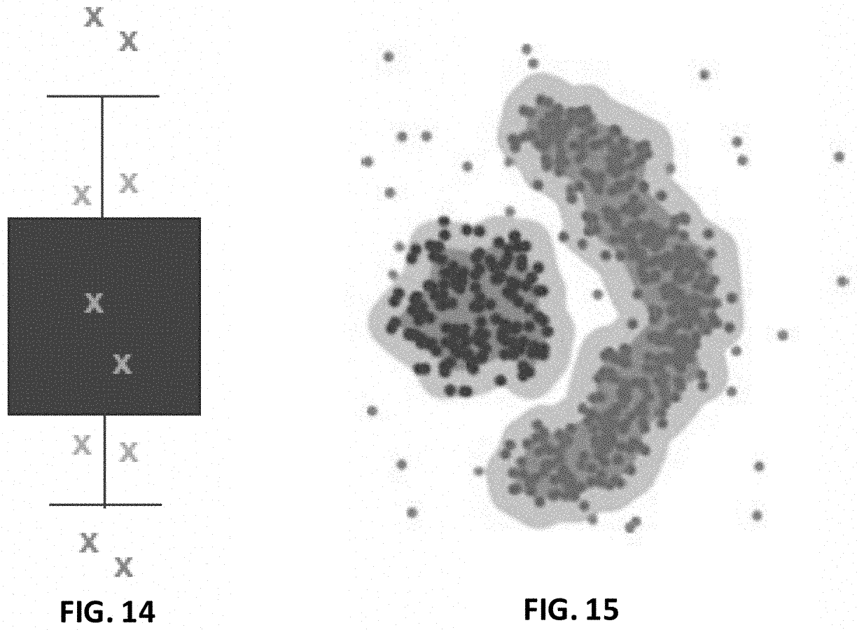

FIG. 14 illustrates a statistical approach for detecting anomalies that uses a boxplot derived from training data. An interquartile range (IQR) may be determined as the middle 50% of the data distribution for the training data. It may be determined that an anomaly has occurred if the data is either greater than the product of k and an upper boundary of the IQR or less than the product of k and a lower boundary if the IQR. The value of the constant K may be established during the training data collection. By way of example and not limitation, the value of K, and thus the threshold for determining anomalies, may be equal to or about 1.5.

FIG. 15 illustrates an unsupervised learning approach for performing a data audit on stored feature data and/or signal samples using a density-based supervised clustering of apps with noise (DBSCAN). DBSCAN is an example of detecting anomalies in stored data using more computationally-intensive anomaly detection techniques, as it performs a data audit using an unsupervised learning approach. DBSCAN creates clusters” around core instance. Anything outside cluster/core instance is outlier. DBSCAN is good at finding non-linearly separable clusters. DBSCAN is efficient in memory and time as it only uses two hyper-parameters.

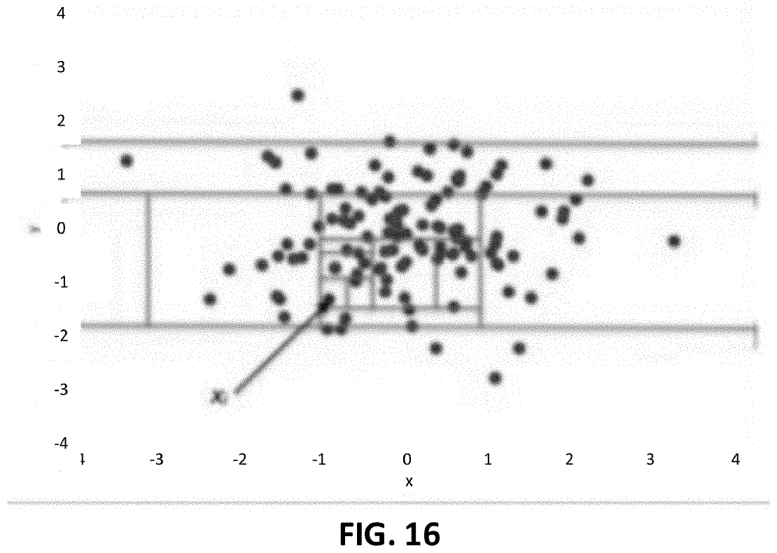

FIG. 16 illustrates an unsupervised learning approach for performing a data audit on stored feature data and/or signal samples using an isolation forest technique. The isolation forest technique randomly splits the dataset into two based on feature, and continues to do so until all instances are isolated. Anomalies are isolated in fewer steps than the remainder of the data. The isolation forest technique is useful for high-dimensional data

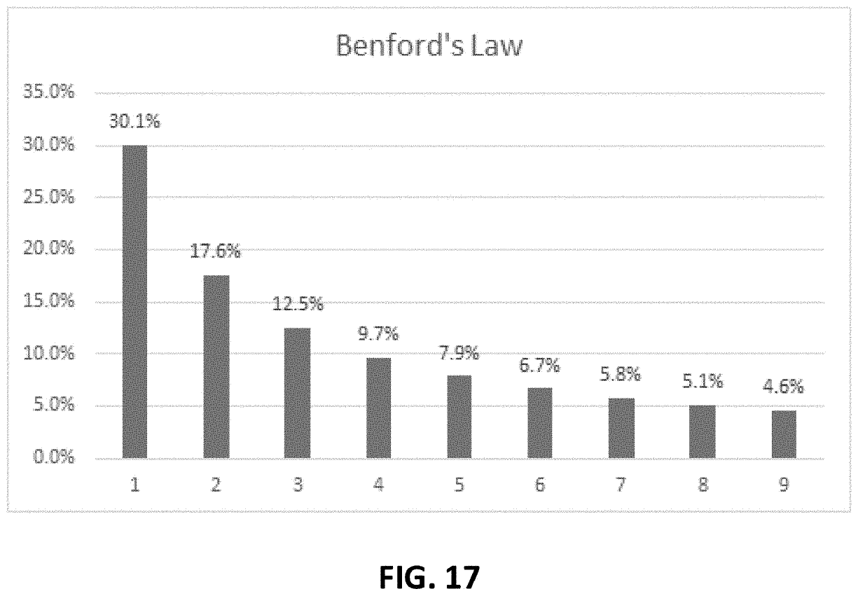

FIG. 17 illustrates a technique based on an empirical law, such as Benford's law, for performing a data audit on stored feature data and/or signal samples. Benford's law indicates that the first digit of every item within a dataset is not random, but rather is logarithmically distributed. Benford's law gives a prediction of the frequency of leading digits using base-10 logarithms where the frequencies decrease as the digits increase from 1 to 9. Thus, the first digit is expected to be “1” about 30.1% of the time, “2” about 17.6% of the time, “3” about 12.5% of the time; 4 about 9.7% of the time, “5” about 7.9% of the time, “6” about 6.7% of the time, “7” about 5.8% of the time, “8” about 5.1% of the time, and “9” about 4.6% of the time. This phenomenon is scale-invariant and base-invariant. Benford's law may be used to detect whether data does not span orders of magnitude and/or has been tampered with in any way.

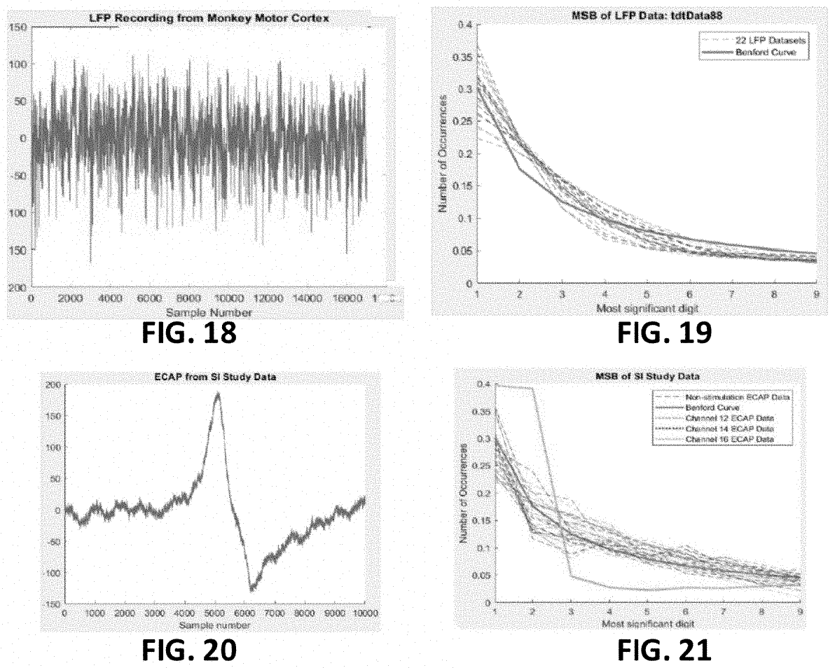

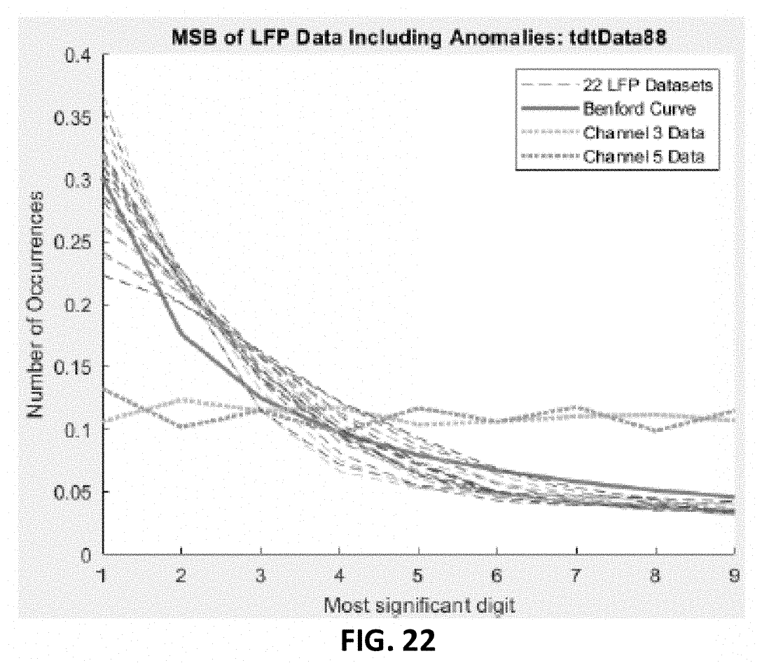

FIGS. 18-22 illustrate, by way of example and not limitation, clinical data following Benford's law. FIG. 18 illustrates a local field potential (LFP) from a monkey's motor cortex; FIG. 19 illustrates the Benford's curve as well as the most significant bit of the LFP data provided in FIG. 18; FIG. 20 illustrates evoked compound action potential (ECAP) data; and FIG. 21 illustrates the Benford's curve as well as the most significant bit for different channels of the ECAP data provided in FIG. 20. FIG. 22 illustrates the most significant bit for different channels of the LFP data. It is noted that the sensed LFP and ECAP data follow's Benford's law, and that contacts that are used for both stimulation and sensing follow a modified Benford's law. However, disconnected channels (e.g., channel 16 in FIG. 21 and channels 3 and 5 in FIG. 22) do not follow Benford's law.

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. However, the present inventors also contemplate examples in which only those elements shown or described are provided. Moreover, the present inventors also contemplate examples using combinations or permutations of those elements shown or described.

Method examples described herein may be machine or computer-implemented at least in part. Some examples may include a computer-readable medium or machine-readable medium encoded with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments may be used, such as by one of ordinary skill in the art upon reviewing the above description. The scope of the invention should be determined with reference to the appended claims, along with the full scope of equivalents to which such claims are entitled.

What is claimed is:

1. A method, comprising:
delivering an electrical therapy using a stimulator operably connected to at least one electrode by delivering an electrical waveform according to waveform parameters;
sensing electrical potentials using sensing circuitry; and
using a controller to automatically perform a process, wherein the automatically performed process includes:
detecting at least one feature in the sensed electrical potentials;
providing closed-loop control of the stimulator using a control algorithm and the detected at least one feature as an input into the control algorithm, wherein the control algorithm defines one or more relationships between at least one feature and the one or more of the waveform parameters, the one or more relationships being determined using feature data;
determining whether the detected at least one feature is anomalous with respect to the feature data used to determine the one or more relationships; and
performing remedial action when it is determined that the at least one feature is anomalous with respect to the feature data.

2. The method of claim 1, wherein the sensing electrical potentials includes sensing local field potentials, evoked compound action potentials (ECAPs), or evoked resonant neural activity (ERNA).

3. The method of claim 1, wherein the sensing electrical potentials includes sensing neural activity or sensing muscle activity.

4. The method of claim 1, wherein the detecting at least one feature includes:
detecting at least one peak, the at least one peak including a minimum peak, a maximum peak, a local minimum peak or a local maximum peak;
detecting an area under a curve;
detecting a curve length;
detecting an oscillation frequency; or detecting a rate of decay for a peak amplitude.

5. The method of claim 1, wherein the providing closed-loop control includes providing closed-loop control based on a feature change for the detected at least one feature with respect to a baseline or a feature difference.

6. The method of claim 1, further comprising implementing unsupervised machine learning techniques to determine whether the detected at least one feature is anomalous with respect to the feature data, wherein the unsupervised machine learning techniques include a density-based supervised clustering of apps with noise (DBSCAN) or an isolation forest.

7. The method of claim 1, wherein the determining whether the detected at least one feature is anomalous includes performing statistical analysis to determine that the detected at least one feature is anomalous with respect to the feature data.

8. The method of claim 7, wherein the detected at least one feature is quantified using digits, and the statistical analysis includes analyzing a most significant digit for the quantified value using Benford's law.

9. The method of claim 7, wherein the statistical analysis includes a Z-score calculated as a difference between a data point for the at least one feature and a mean of the training data, wherein the mean is divided by a standard deviation of the training data, wherein the at least one feature is determined to be anomalous when the Z-score exceeds a threshold.

10. The method of claim 7, wherein the statistical analysis includes a boxplot derived from training data, wherein the at least one feature is determined to be anomalous when a data point for the at least on feature is great than or less than a factor of an upper limit for an interquartile range or a factor of a lower limit for the interquartile range.

11. The method of claim 1, wherein the determining whether the detected at least one feature is anomalous is performed before detecting a subsequence instance of the at least one feature in the sensed evoked signal.

12. The method of claim 1, wherein the detected at least one feature is determined to be not anomalous before adjusting at least one waveform parameter based on the detected at least one feature.

13. The method of claim 1, further comprising storing a plurality of instances of the detected at least one feature, and auditing the plurality of instances to determine if any one or more of the instances correspond to anomalous detected at least one feature.

14. The method of claim 1, wherein the performing remedial action includes automatic and/or manual processes for:
disabling or adjusting the closed-loop control;
reconfiguring a sensing configuration; or
reconfiguring the feature data used to determine the one or more relationships between the at least one feature and the one or more waveform parameters.

15. The method of claim 1, wherein the performing remedial action includes communicating with the patient to troubleshoot or to send an encrypted report.

16. A non-transitory machine-readable medium including instructions, which when executed by a machine, cause the machine to perform a method comprising: delivering an electrical therapy by delivering an electrical waveform according to waveform parameters;
sensing electrical potentials; and
automatically perform a process, wherein the automatically performed process includes:
detecting at least one feature in the sensed electrical potentials;
providing closed-loop control of the stimulator using a control algorithm and the detected at least one feature as an input into the control algorithm, wherein the control algorithm defines one or more relationships between at least one feature and the one or more of the waveform parameters, the one or more relationships being determined using feature data;
determining whether the detected at least one feature is anomalous with respect to the feature data used to determine the one or more relationships; and
performing remedial action when it is determined that the at least one feature is anomalous with respect to the feature data

17. The non-transitory machine-readable medium of claim 16, wherein the detected at least one feature is quantified using digits, and the statistical analysis includes analyzing a most significant digit for the quantified value using Benford's law.

18. The non-transitory machine-readable medium of claim 16, wherein the statistical analysis includes a Z-score calculated as a difference between a data point for the at least one feature and a mean of the training data, wherein the mean is divided by a standard deviation of the training data, wherein the at least one feature is determined to be anomalous when the Z-score exceeds a threshold.

19. The non-transitory machine-readable medium of claim 16, wherein the statistical analysis includes a boxplot derived from training data, wherein the at least one feature is determined to be anomalous when a data point for the at least on feature is great than or less than a factor of an upper limit for an interquartile range or a factor of a lower limit for the interquartile range.

20. A system, comprising:
a stimulator operably connected to at least one stimulation electrode, and configured to deliver an electrical therapy using the at least one electrode by delivering an electrical waveform according to waveform parameters;
sensing circuitry operably connected to at least one sensing electrode, and configured to sense electrical potentials;
a controller operably connected to the stimulator and the sensing circuitry, wherein the controller is configured to:
detect at least one feature in the sensed electrical potentials;
provide closed-loop control of the stimulator using a control algorithm and the detected at least one feature as an input into the control algorithm, wherein the control algorithm defines one or more relationships between at least one feature and the one or more of the waveform parameters, the one or more relationships being determined using feature data;
determine whether the detected at least one feature is anomalous with respect to the feature data used to determine the one or more relationships; and
perform remedial action when it is determined that the at least one feature is anomalous with respect to the feature data.
